(19) United States
(12) Patent Application Publication
(10) Pub. No.: US 2010/0152298 A1
(43) Pub. Date: Jun. 17, 2010

(54) Present invention relates to a treatment for the management of cancer more specially the use of Flutamide anti-androgen drugsa

(75) Inventor: Fatma Jassab Faleh Al-Saeedi, Safat (KW)

Correspondence Address: FURR LAW FIRM, 2622 DEBOLT ROAD, UTICA, OH, 43080, US

(73) Assignee: UNIVERSITY OF KUWAIT, Safat (KW)

(21) Appl. No.: 12/029,473

(22) Filed: Feb. 12, 2008

(51) Int. Cl. A61K 31/167; A61P 35/00

(52) U.S. Cl. 514/629

(57) ABSTRACT

This invention helps in the treatment and the management of cancer. It is the use of flutamide drug to treat and manage cancer where the Flutamide anti-androgen drug acts in another mechanism other than the well know androgen respects (AR) mechanism.

### FIELD OF THE INVENTION

The present invention relates to a treatment for the management of cancer more specially the use of Flutamide anti-androgen drugs where Flutamide anti-androgen drug acts in another mechanism other than the well know androgen respects (AR) mechanism.

### BACKGROUND OF THE INVENTION

Cancer is a deadly disease. Even today, there exists a need for better tools and abilities to treat it.

### Prior Art Reference and Discussion

U.S. Pat. No. 6,479,063 by Weisman, et al. and issued on Nov. 12, 2002, is for a Therapeutic uses of hormonal manipulation using combinations of various agents to treat atherosclerosis. It discloses a method of decreasing atherosclerosis and its complications involving administering to a human or animal various combinations of medications with Finasteride, Bicalutamide, Flutamide and Nilutamide.

U.S. Pat. No. 6,228,401 by James, et al. and issued on May 8, 2001, is for Processes for preparing flutamide compounds and compounds prepared by such processes. It discloses a process for preparing compounds comprising flutamide API having a specific surface area of at least 0.35 m.sup.2 /cm.sup.3 which comprise blending pharmaceutically acceptable diluents with unmilled flutamide API and milling the blended material until the specific surface area is at least 0.35 m.sup.2 /cm.sup.3. The present invention also provides compounds prepared by such processes.

U.S. Pat. No. 6,187,345 by James, et al. and issued on Feb. 13, 2001, is for Flutamide compositions and preparations. It discloses flutamide, having a range of certain particle sizes and specific surface area, and methods for preparing such flutamide which are useful for preparing pharmaceutical formulations for the treatment of prostatic carcinoma and benign prostatic hypertrophy. The present invention further provides such compositions and methods of treating such disease states.

U.S. Pat. No. 5,994,362 by Gormley, et al. and issued on Nov. 30, 1999, is for a Method of treatment for prostatic cancer. It discloses a new treatment for men with prostatic cancer involving combination therapy of a 5.alpha.-reductase inhibitor, i.e., a 17.beta.-substituted 4-azasteroid, a 17.beta.-substituted non-azasteroid, 17.beta.-acyl-3-carboxyandrost-3,5-diene, benzoylaminophenoxybutanoic acid derivative, fused benz(thio)amide or cinnamoylamide derivative, aromatic 1,2-diethers or thioethers, aromatic ortho acylaminophenoxy alkanoic acids, ortho thioalkylacylamino-phenoxy alkanoic acids, pharmaceutically acceptable salts and esters thereof, and particularly finasteride, in combination with an antiandrogen, i.e. flutamide. Pharmaceutical compositions useful for treatment are also disclosed.

U.S. Pat. No. 5,023,234 by Labrie and issued on Jun. 11, 1991, is for a Combination male breast cancer therapy. It discloses a method of treatment of breast cancer in susceptible male animals whose testicular hormonal secretions are blocked by surgical or chemical means, e.g., by use of an LH-RH agonist, e.g., [D-Trp.sup.6, des-Gly-NH.sub.2.sup.10]LH-RH ethylamide which comprises administering an antiandrogen, e.g., flutamide and optionally at least one inhibitor of sex steroid biosynthesis, e.g., aminoglutethimide and/or ketoconazole. Pharmaceutical compositions useful for such treatment are also disclosed.

U.S. Pat. No. 4,921,941 by Nagabhushan, et al. and issued on May 1, 1990, is for Orally active antiandrogens. It discloses Antiandrogenic peptidyl esters, particularly tri-peptidyl esters, of the active metabolite of flutamide.

U.S. Pat. No. 4,666,885 by Labrie and issued on May 19, 1987, is for a Combination therapy for treatment of female breast cancer. It discloses a method of treatment of breast cancer in susceptible animals whose ovarian hormonal secretions are blocked by surgical or chemical means, e.g., by use of an LH-RH agonist, e.g., [D-Trp.sup.6, des-Gly-NH.sub.2.sup.10]LH-RH ethylamide with a therapy comprising administering an antiandrogen, e.g., flutamide and an optionally, an inhibitor of adrenal sex steroid biosynthesis e.g., aminoglutethimide, pharmaceutical compositions useful for such treatment and two, four and five component pharmaceutical kits containing such compositions.

U.S. Pat. No. 4,659,695 by Labrie and issued on Apr. 21, 1987, is for a Method of treatment of prostate cancer. It discloses a method of treatment of prostate cancer in susceptible male animals including humans whose testicular hormonal secretions are blocked by surgical or chemical means, e.g., by use of an LH-RH agonist, e.g., [D-Trp.sup.6, des-Gly-NH.sub.2.sup.10]LH-RH ethylamide which comprises administering an antiandrogen, e.g., flutamide in association with at least one inhibitor of sex steroid biosynthesis, e.g., aminoglutethimide and/or ketoconazole. Pharmaceutical compositions useful for such treatment and three, four and five component kits containing such compositions are also disclosed.

U.S. Pat. No. 4,474,813 by Neri, et al. and issued on Oct. 2, 1984, is for Pharmaceutical preparations comprising flutamide. It discloses a pharmaceutical preparation comprising flutamide, useful in the treatment of prostatic carcinoma. It is assigned to Schering Corporation.

### SUMMARY OF THE INVENTION

This invention helps in the treatment and the management of cancer. It is the use of a flutamide drug to treat and manage cancer where the Flutamide anti-androgen drug acts in another mechanism other than the well know androgen respects (AR) mechanism.

### BRIEF DESCRIPTION OF THE DRAWINGS

The above and other aspects, features, and advantages of the present invention will be better and more fully understood by those skilled in the art with reference to the following detailed and more particular description of specific and preferred embodiments thereof, presented in conjunction with the following drawings to show how the same may be carried into effect, wherein:

FIG. 1 shows the chemical structure for Flutamide;

FIG. 2 a Table showing S phases of PC-3 cells after exposure; and

FIG. 3 displays the ability of flutamide drug to act on cancer cell.

### DETAILED DESCRIPTION OF BEST MODE FOR CARRYING OUT THE INVENTION

There will now be described, by way of example only, the best mode contemplated by the inventor for carrying out the invention. In the following description numerous specific details are set forth in order to provide a thorough understanding of the present invention. It will be apparent however, to one skilled in the art, that the present invention may be practiced without limitation to these specific details. In other instances, well known methods and structures have not been described in detail so as not to unnecessarily obscure the present invention.

The present invention relates to a treatment for the management of cancer more specially the use of Flutamide anti-androgen drugs. This invention helps in the treatment and the management of cancer. It is the use of flutamide drug to treat and manage cancer in mammals where the Flutamide anti-androgen drug acts in another mechanism other than the well know androgen respects (AR) mechanism. That flutatmide drug kills and destroys cancerous cells with no androgen receptors. The drug does not depend on or contain androgen and androgen receptors.

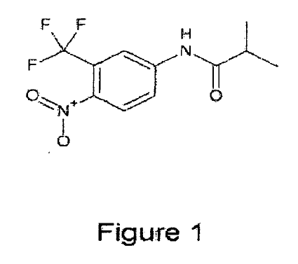

Flutamide is an oral antiandrogen drug. Its chemical compisition is shown in FIG. 1. It normally taken orally but can be admistered in other ways.

Unlike the hormones with which it competes, flutamide is not a steroid; rather, it is a substituted anilide. After absorption, the molecule is quickly α-hydroxylated to its primary active form, hydroxyflutamide. Flutamide is excreted in various forms in the urine, the primary form being 2-amino-5-nitro-4-(trifluoromethyl) phenol.

[Methyl-¹¹C]—choline positron emission tomography (PET) was used to image many types of cancers especially prostate cancer. The incorporation of [Methyl-¹¹C]—choline into breast tumors (MCF-17) cells correlated strongly with proliferation as determined by [Methyl-¹¹C]—thymidine uptake. Chemotherapy-induced modulation in [Methyl-¹¹C]—choline incorporation in MCF-7 cells treated with 5-Fluorouracil (5-FU) in certain mechanism. The pure anti-androgen drug, flutamide killed androgen-independent prostate cancer PC-3 cells. Mechanisms responsible for the effect of flutamide on [Methyl-³H]—choline incorpation into PC-3 tumor cells treated with flutamide were reported.

The effect of choline incorporation on PC-3 cells treated with a range of doses of flutamide inhibiting growth by 20-70% was studied. Treated and control cells were incubated with [Methyl-¹¹C]—choline for 10 minutes, then in non-radioactive medium to simulate the rapid block blood clearance of [Methyl-¹¹C]—choline tracer, and then extracted with organic and aqueous solvents to determine the intracellular distribution of the tracer.

The results are the PC-3 cells proliferation was inhibited by flutamide for 3 days resulted in a build up of cells in S phase at 10 nM and [Methyl-³C]—choline incorporation per a cell was found to be decreased in traest compared to untreated cells.

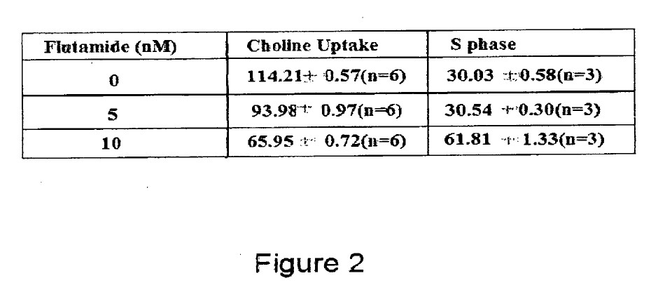

FIG. 2 shows a table where [Methyl-³C]—choline incorporation (n=6) and corresponding S phases (n=3) of PC-3 cells after an exposure to 0.5, and 10 nM flutamide. The data is express as the mean d.p.m/Up protein.

The results show that as the concentration of the drug increases, there is a significant reduced uptake of choline in cancer cells. This invention demonstrate that a decrease in cell viability, as well as an accumulation of cells in the S phase (NA synthesis) of the cell cycle after the treatment of PC-3 cells with flutamide. The results reflect the importance of this the treatment of management of cancer.

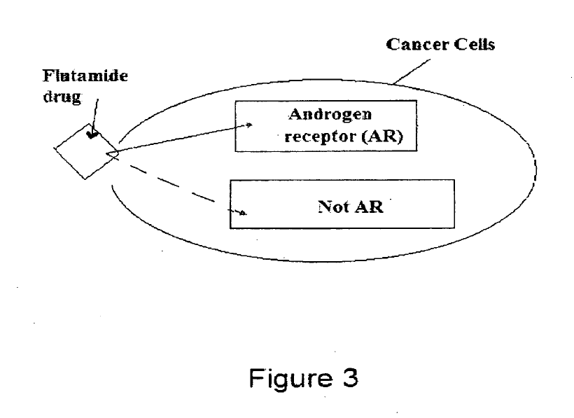

FIG. 3 displays the ability of flutamide drug to act on and kill a cancer cells via a mechanism that does not depend on androgen receptors.

Equivalents

From the foregoing description, one skilled in the art can easily ascertain the essential characteristics of this invention and, without departing from the spirit and scope thereof, can make various changes and modifications of the invention to adapt it to various usages and conditions. Such variations and changes may include, for example, altering the number of components in the housing or using equivalents. It is believed that such can be accomplished without excessive experimentation. In any case, any such variations are all claimed under the scope of this invention.

The methods of the present invention have been explained with reference to plurality of references the teachings of which are all incorporated herein by reference.

From the foregoing description, one skilled in the art can easily ascertain the essential characteristics of this invention and, without departing from the spirit and scope thereof, can make various changes and modifications of the invention to adapt it to various usages and conditions. Such variations and changes may include, for example, altering the number of components in the housing or using equivalents. It is believed that such can be accomplished without excessive experimentation. In any case, any such variations are all claimed under the scope of this invention.

We claim:

1. A process to treat cancer in mammals comprising:
using a Flutamide anti-androgen drug without depending on or containing androgen and androgen receptors.

2. A process according to claim 1 further comprising:
having said drug being take orally.

3. A process to treat cancer in mammals comprising:
having a Flutamide anti-androgen drug acting in another mechanism other than the androgen respects (AR) mechanism

4. A process according to claim 3 further comprising:
having said drug being take orally.
